(19) United States
(12) Patent Application Publication
(10) Pub. No.: US 2007/0287892 A1
(43) Pub. Date: Dec. 13, 2007

(54) Detection, measurement, and communication of physiological properties, and generation and communication of activation signals in response to physiological properties

(76) Inventor: Hector Jose Estrella, Santo Domingo (DO)

Correspondence Address: MALLOY & MALLOY, 2800 S.W. THIRD AVENUE, HISTORIC CORAL WAY, MIAMI, FL, 33129, US

(21) Appl. No.: 11/498,941

(22) Filed: Aug. 3, 2006

Related U.S. Application Data
(63) Continuation-in-part of application No. 11/109,499, filed on Apr. 19, 2005.
Application No. 11/109,499 is a continuation-in-part of application No. 10/418,518, filed on Apr. 18, 2003, now abandoned.

(51) Int. Cl. A61B 5/00

(52) U.S. Cl. 600/300

(57) ABSTRACT

A system for the detection, measurement, and communication of a value of one or more physiological property of a person, or other living organism, includes a sensor assembly structured to detect, measure, and communicate at least one value during a monitoring period. The sensor assembly may include an array of interchangeable sensors structured to detect, measure, and communicate such values during the monitoring period. The value or values of the physiological property or properties may be communicated to a central processing unit via a communication interface wherein the value(s) are analyzed to generate an activation signal structured to affect operation of an integrated component. Alternatively, the value or values may be communicated to one or more third party, such as a physician, psychologist, security personnel, friend, family member, intimate partner, etc., for independent review and analysis. A method is also provided for detecting, measuring, and communicating one or more value, and generating and communicating an activation signal in response thereto.

### CLAIM OF PRIORITY

The present application is a Continuation-In-Part patent application of previously filed, currently pending U.S. patent application having Ser. No. 11/109,499 and a filing date of Apr. 19, 2005, which is a Continuation-In-Part of U.S. patent application having Ser. No. 10/418,518 and a filing date of Apr. 18, 2003, now abandoned.

### BACKGROUND OF THE INVENTION

1. Field of the Invention

The present invention relates to a system for the detection, measurement, and communication of the value of one or more physiological property of person, or other living organism, the physiological property being monitored by a sensor assembly disposed in a communicating relation with a communication interface. In at least one embodiment, the communication interface is structured to analyze one or more physiological property values, and to generate at least one activation signal in response thereto, wherein the activation signal is structured to affect the operation of one or more integrated component.

2. Description of the Related Art

Through the ages, various devices, systems, and methods have been developed to monitor a variety of physiological properties of a person. The interest in monitoring such properties often stem from medical necessity or medical research in hopes of determining the impact of certain ailments, afflictions, and/or other stimuli on one or more bodily functions of a person. For example, during even the most routine visit to a medical doctor, certain physiological properties or bodily functions are almost always measured and noted in a patient's file before the doctor ever sees the patient, such as blood pressure, temperature, and pulse rate. This is due in part to the fact that the normal values of these particular bodily functions are well established, and a deviation in one or more of these parameters may be indicative of a specific ailment or affliction. It is further know that abrupt changes in certain physiological properties or bodily functions may be indicative of certain stimuli to which the patient has been exposed and/or sensations a person is experiencing.

Given the significance of certain bodily functions as an indicator of a person's health and/or present state of being, a number of devices have been developed to permit one or more of these functions to be readily measured and/or monitored. For example, one such diagnostic device comprises a glove member structured to fit on a user's hand which is interconnected via a transmission cable to an interface/command center. The glove member includes a variety of diagnostic sensors structured to measure such parameters as EKG, blood pressure, pulse rate, and/or temperature. This device may also be structured to allow measurement of blood oxygen levels as well as the detection of sound waves generated by the patient's heart and lungs when the glove member is positioned over the chest of the user, via an auscultation device. The interface/command center is structured to transmit the data collected to a remote location, for example, via an internet connection, wherein the data is interpreted by trained personnel, such as a doctor or nurse, who then provide medical advice and/or instruction to the user.

While the foregoing device provides a means for a person to measure the values of a variety of bodily functions, the device requires the person to be physically interconnected to the interface/command center via the cable, which limits use of the device to locations where such an interface/command center is located, and further limits the data collected to the brief period of time in which the person is physically interconnected. Additionally, while the aforementioned device permits the collection of data corresponding to the various bodily functions, it requires trained personnel to interpret the data collected before it is of value to the user.

A number of portable devices have also been developed which allow a user to measure various bodily functions. One such device permits the user to monitor such bodily functions as blood pressure, temperature, and pulse rate, and includes a display screen to provide the user with real time data. This device also allows for the storage of data collected over a period of time for retrieval and analysis at a later time, such as may be requested or required by trained medical personnel.

Other portable devices include an electrocardiogram monitor which allow a user to monitor their electrocardiogram on a viewable screen, and a cardiac meter which is structured to be worn on a user's wrist. Such portable devices afford a user a degree of privacy while monitoring certain bodily functions, and it is known that monitoring of bodily functions in private may be desirable in certain instances. For example, while monitoring the bodily functions of a person engaged in sexual activity, certain person's may prefer or in fact insist that such monitoring be conducted privately. Of course, the monitoring and interpretation of certain physiological parameters of persons engaged in sexual activity, and particularly the involuntary or uncontrollable changes in certain bodily functions which occur during orgasm, are believed to provide useful information which may assist persons in understanding and improving sexual techniques, thereby providing them with the benefits of a more fulfilling sex life.

At least one device has been developed to identify a contraction of certain muscles in an attempt to determine the occurrence of an orgasm of the user. Specifically, the device is structured to identify the contraction of the anal sphincter and associated muscles of the person being monitored. While it appears that this device may be utilized in private by the user, the device must be inserted into the anal canal, which many users may find overly intrusive and/or uncomfortable, particularly while engaging in sexual activity, thereby precluding widespread usage and benefit.

As such, it would be beneficial to provide a system which may be utilized in private by a user to monitor one or more bodily functions while the person is engaged in various activities, such as sexual activity. It would further be helpful if such a system allowed the bodily functions to be monitored in a non-intrusive manner, to assure widespread acceptance and usage of any such system. Additionally, such a system would preferably provide the user with results which are interpreted without requiring the assistance of specially trained personnel and/or equipment. More specifically, it would be beneficial to provide a system which allows a user to monitor and detect uncontrollable changes in certain bodily functions which are indicative of a particular sensation a person is experiencing, such as fear or an orgasm. In particular, it would be preferable to provide a system which a user may utilize in a non-intrusive, private manner for the detection of an orgasm. It would further be beneficial to provide a method which a user may utilize in a non-intrusive, private manner for the detection of an orgasm. A further benefit would be to monitor and detect baseline values for certain bodily functions.

It would also be beneficial to provide a system for detecting, measuring, and communicating a value of at least one physiological property of a person, via a sensor assembly disposed in a communicative relation with a communication interface. Also it would be helpful if the communication interface of such a system was structured to communicate with one or more integrated component and/or a third party monitoring device. Additionally, it would be preferable for the communication interface to be structured to generate at least one activation signal and to communicate the activation signal to at least one integrated component, and to affect the operation of the same. A further benefit may be realized by providing a system wherein the communication interface is structured to communicate one or more physiological property value to a third party monitoring device, as well as to receive one or more physiological property value from one or more third party device, such as, by way of example, one or more additional systems.

### SUMMARY OF THE INVENTION

As indicated above, at least one embodiment of the present invention is directed to a system for detecting an uncontrollable change in a person's physiology. It will be appreciated from the following disclosure that in at least one other embodiment, the present invention may be utilized to detect more than one uncontrollable change, for example, a series of uncontrollable changes, and the system may even be utilized to detect more than one series of uncontrollable changes. The system comprises a sensor assembly which is disposed in a monitoring relation with the person. More in particular, the sensor assembly comprises at least one sensor structured to monitor at least one preselected bodily function of the person during an activity monitoring period, however, in at least one embodiment, the sensor assembly comprises a plurality of sensors structured to monitor a plurality of bodily functions. The sensor assembly may be physically attached to one or more portions of the person's body, or it may be structured to monitor the bodily function or functions of the person remotely, such as, by way of example only, via infrared, microwave, optical, sound or light waves, or other remote signal.

The sensor assembly may be structured to monitor one or more of a plurality of bodily functions including, but not limited to blood pressure, pulse, temperature, respiration, muscular contractions, caloric expenditure, blood oxygen level, release of sweat and/or other components such as endorphins, pheromones, adrenaline, etc., as well as sound waves generated either vocally or via the pulse, heart beat, and/or respiration of the person being monitored.

Additionally, the present invention also includes a central processing unit which is disposed in a communicating relation with the sensor assembly, which is to say that the sensor assembly is structured to transfer at least one activity data set to the central processing unit corresponding to the at least one preselected bodily function being monitored during the activity monitoring period. In at least one embodiment, however, the sensor assembly is structured to transfer a plurality of activity data sets, each corresponding to a different one of the preselected bodily functions being monitored during the activity monitoring period. In yet another embodiment, the sensor assembly may be structured to transfer a plurality of activity data sets, each corresponding to a different one of the preselected bodily functions being monitored during each of a different one of a plurality of activity monitoring periods.

Also in accordance with the present invention, the central processing unit is structured to interpret each activity data set transferred by the sensor assembly and to detect a predetermined change in the preselected bodily function to which each activity data set corresponds. Also, the system may include an output device which is operatively associated with the central processing unit and is structured to generate an indication upon detection of the predetermined change in one or more of the preselected bodily functions, by the central processing unit.

In at least one embodiment of the system of the present invention, the central processing unit comprises a data memory module structured to store at least one, but preferably a plurality of activity data sets, each corresponding to one of the plurality of bodily functions monitored during each of a plurality of activity monitoring periods. The data memory module is further structured to permit the central processing unit to correlate each of the plurality of activity data sets. In one further embodiment, the central processing unit comprises an external data transmission port structured to transmit the plurality of activity data sets to an external data processing device to provide additional storage and facilitate further analysis of the plurality of activity data sets.

The system of the present invention, in at least one embodiment, is utilized to measure an uncontrollable change in the physiology of other living creatures including, but not limited to dogs, cats, mice, rats, birds, and horses, among others.

The bodily functions and/or parameters which may be obtained and/or monitored utilizing the system of the present invention may further include shockwaves, electrowaves, brainwaves, and/or the aura generated by the person. In one further embodiment, the system may utilize hair recognition technology to detect the size, diameter and/or length, density, color, etc, of the hair located on a portion of the person's body.

In addition, the system may comprise the measurement of geometrical and special dimensions of various portions of the person's body including, but not limited to measurement of the person's nose, ears, eyes, penis, or vagina, just to name a few.

One further embodiment of the present invention provides for the measurement and monitoring of the amount of heat absorbed or heat released by the person's body. Further, the system may be utilized to measure and monitor fluids, hormones, proteins, gases, odors, particulate matter, sounds and/or sound waves expelled from or absorbed by the person's body, including macro-, micro-, and/or nano-measurement of the same. At least one other embodiment of the system may be utilized for the detection of any contraction of the body, including contractions of the muscles and/or organs, as well as any voluntary and/or involuntary movement or contraction of the body, such as may be detectible by a motion sensor or otherwise. As one example, a sensor may be temporarily inserted into one or more body cavity of a person, and the frequency and magnitude of the contraction of the muscles at the periphery of the body cavity or body cavities may be detected and measured via the present system. Yet one further embodiment of the present invention provides for detection and measurement of a person's weight as another factor for consideration in evaluating the data obtained form a user of the system.

The system of the present invention may also be utilized to monitor, measure, and calculate the amount, concentration, rate of absorption or adsorption, rate of expulsion, degree of contraction, and/or frequency (where applicable) of at least the physical parameters identified herein. Any number of the physical parameters identified herein may also be present and monitored in the environment surrounding the person being monitored.

One other embodiment of the present invention may be utilized to monitor skin color, hair color, stains, physiological and/or biological changes in the body due to illness or external stimuli. Yet another embodiment may be utilized to monitor for galvanic skin response, the galvanic skin response being a tiny and rapid fluctuation in the skin moisture which may be associated with arousal, or possible illness, wherein a compact palm sensor may be utilized to measure for such a galvanic skin response. The values of any the parameters detected, monitored, stored, and/or analyzed by at least one embodiment of the system of the present invention may be expressed in terms of percentages, exact amounts, estimated or approximated amounts, and/or scaled values. In one further embodiment, the system has storage capability for the baseline and activity data sets, the baseline and activity biometric parameters, and any calculated values generated utilizing the same, which may be stored and/or transmitted by the system.

At least one embodiment of the system of the present invention includes capacity for wired and/or wireless data transmission for receiving and/or transmitting data to or from external devices including, but not limited to, external processing units and/or external output devices. More specifically, the system is capable of exchanging data with any external equipment or mechanism.

Additionally, the sensor assembly in at least one embodiment may utilize laser, infrared, ultraviolet, or other electromagnetic radiation waves, magnetic, non-magnetic, electrical, audible, or visual energy to effect wireless data and/or signal transmission between components of the system.

One further embodiment of the present invention may be comprise a secure activation mechanism utilizing a biometric identification device to measure any of a variety of biometric parameters including, but not limited to, fingerprint patterns, iris patterns, voice and/or other sound patterns, wave energy patterns emitted from the body, hair patterns, including density, color, size, i.e. diameter and/or length, skin porosity patterns, tattoos, geometrical and spatial dimensions of portions of the person's body, and respiration patterns, just to name a few. Other biometric parameters may include biological secretions from the body, and energy waves generated by the body, for example, the person's aura.

The system of the present invention may further employ barcodes, magnetic, and/or non-magnetic systems to secure access and activation of the system. The system may further provide for encryption and decryption of the data received, transmitted, stored, analyzed, or otherwise utilized by the system.

Yet one further embodiment of the system of the present invention comprises internal or external global positioning technology, cellular and non-cellular communications capacity, and is structured to store and play any media which may be scanned and/or read into the system in any data format. As noted below, one embodiment of the present invention employs at least one detector or sensor which is physically attached to or implanted in a person's body, for example, on a tooth, in the ear canal, via a piercing, under the skin, etc., and as such, the sensor or detector may be combined with the global positioning technology such that the sensor or detector also serves to provide a means to positively identify the location of any particular user of the system.

In addition to the system described above, the present invention also provides a method for detecting an uncontrollable change in a person's physiology. Specifically, the method comprises monitoring at least one preselected bodily function of the person during an activity monitoring period, and transferring an activity data set corresponding to the preselected bodily function for analysis. At least one embodiment of the present invention provides for monitoring a plurality of preselected bodily functions during the activity monitoring period.

The method also provides for interpreting one or more one activity data sets by comparing each activity data set to a table containing a value or range of values of a predetermined change in one or more preselected bodily functions being monitored and detecting a predetermined change in the preselected bodily function to which each activity data set corresponds. In at least one embodiment, the method comprises interpreting each of the plurality of activity data sets via a central processing unit and detecting the predetermined change. Additionally, the method of the present invention may comprise generating an indication upon detection of the predetermined change in the at least one preselected bodily function.

Yet another embodiment of the present invention comprises a system for the detection, measurement, and communication of one or more physiological property such as may be monitored, detected, and measured from a person or another living organism. As in the previously described embodiments, the system of the present embodiment comprises a sensor assembly which is disposed in a monitoring relation with a person or other subject, for example, another living organism such as a dog, cat, livestock, etc. Also, as above, the sensor assembly of the system of the present embodiment includes at least one sensor, the sensor being structured to detect and measure a value of at least one preselected physiological property of a person, or other living organism, during one or more monitoring period.

Additionally, the system of the present embodiment includes a communication interface which is disposed in a communicating relation with the sensor assembly, wherein the sensor assembly is structured to communicate the value of at least one preselected physiological property which is detected and measured during the monitoring period to the communication interface. In at least one embodiment, the communication interface includes a central processing unit, such as in the previously disclosed embodiments of the present invention, the central processing unit being disposed in a communicating relation with the sensor assembly. At least one embodiment further comprises an input interface to the communication interface and/or central processing unit such as a computer keyboard, mouse, voice controller, to permit a user to setup, configure, or otherwise direct and/or modify operation of the system based upon user necessitated or desired operating parameters.

In the present embodiment, the central processing unit is structured to analyze the value of the at least one preselected physiological property, and to generate at least one activation signal which corresponds to a value of a preselected physiological property. As above, it is understood to be within the scope and intent of the present invention for the sensor assembly to monitor for and detect and measure a value of each of a plurality of physiological property during one or more monitoring period. As such, the central processing unit of the present embodiment of the system may be further structured to analyze each of the plurality of values of each of the plurality of physiological properties obtained during the monitoring period, and to generate one or more activation signals corresponding to each of the plurality of values.

The communication interface, in at least one embodiment of the present invention, is further disposed in a communicating relation with at least one integrated component. The integrated component may comprise an environmental control device, a personal control device, a security control device, or any other of a plurality of devices whose operation may be affected by one or more activation signal. More in particular, in the present embodiment of the system the communication interface is disposed in a communicating relation with at least one integrated component and is further structured to communicate at least one activation signal to the at least one integrated component, thereby affecting the operation of the at least one integrated component.

A further embodiment of the system for the detection measurement and communication of physiological properties includes at least a first biometric identification device which is structured to measure at least one biometric parameter so as to positively identify a person or subject to be disposed in a monitoring relation with a sensor assembly. In this embodiment, the communication interface is further structured and disposed in a communicating relation with a third party monitoring device. More in particular, the communication interface in this embodiment is structured to transmit each value of one or more preselected physiological properties obtained during a monitoring period to the third party monitoring device.

In order to protect against unwanted or unauthorized interception and monitoring of a subject's physiological properties via a third party monitoring device, at least one embodiment of the present invention further comprises a second biometric identification device which is structured to measure at least one biometric parameter to positively identify an observer prior to permitting access to the third party monitoring device, thereby permitting access to the preselected physiological property values of the person or subject disposed in a monitoring relation with the sensor assembly. In at least one embodiment, a third party monitor may be provided to a parent or guardian of a child, such that the parent or guardian can remotely monitor their child or charge, and to identify any abnormal values which may be indicative of a problem, such as, injury, fear, abuse, movement from the home or classroom, etc. Once again, a biometric identification device may be utilized to prevent unauthorized access to a minor child's physiological property data, the biometric device being utilized to limit access to the child's parent(s) and/or guardian(s).

In yet one further embodiment, a biometric identification device may be utilized to require authentication and authorization prior to communication of any physiological data to any communication interface, such as, by way of example only, requiring a positive biometric identification to “lock” the sensor assembly in a monitoring disposition with a user, prior to permitting the transmittal of any physiological data. In a further embodiment, unauthorized “unlocking” of the device may also be controlled via a biometric identification device, and the system may further be configured to signal an alarm to indicate that an unauthorized “unlocking” has occurred, for example, when someone without authority removes a sensor assembly from a minor child being monitored with the present system.

A method for detecting, measuring, and communicating physiological properties, in conjunction with the above referenced system, comprises the following steps. To begin, the method comprises a step of monitoring a person or subject with the sensor assembly during a baseline monitoring period. The present method also includes detecting one or more preselected physiological property via the sensor assembly during the baseline monitoring period, and measuring a baseline value of the one or more physiological property during said baseline monitoring period. The method also includes communicating the baseline value or values of the one or more physiological property measured during the baseline monitoring period to a communication interface.

Once the baseline values of one or more physiological property have been detected, measured, and communicated to the communication interface, the present method further includes monitoring the person with the sensor assembly during an active monitoring period. Once again, as in the baseline monitoring period, the present invention includes detecting one or more preselected physiological property via the sensor assembly during the active monitoring period, and measuring an active value of one or more preselected physiological property which is detected during the active monitoring period. Also, the present method comprises communicating the active value or values of the one or more preselected physiological property measured during the active monitoring period to the communication interface.

After obtaining the baseline and active values of one or more preselected physiological property and communicating the same to the communication interface, the method of the present invention comprises analyzing the baseline value and the corresponding active value for each of the preselected physiological properties detected during the baseline and active monitoring periods. Finally, the present method includes generating at least one activation signal wherein the activation signal is at least partially defined by a predetermined variance between a baseline value and an active value of at least one of the plurality of preselected physiological properties.

At least one further embodiment of the method of the present invention comprises communicating at least one activation signal to an integrated component, thereby affecting the operation of the same, as described above.

These and other objects, features and advantages of the present invention will become more clear when the drawings as well as the detailed description are taken into consideration.

### BRIEF DESCRIPTION OF THE DRAWINGS

For a fuller understanding of the nature of the present invention, reference should be had to the following detailed description taken in connection with the accompanying drawings in which:

FIG. 1 is a schematic view of one embodiment of a system of the present invention.

FIG. 2 is a schematic view of one embodiment of a sensor assembly of the system of the present invention.

FIG. 3 is a schematic view of one other embodiment of a sensor assembly of the system of the present invention.

FIG. 4 is a schematic view of yet another embodiment of a sensor assembly of the present invention.

FIG. 5 is a side elevation of one embodiment of an output device of the present invention.

FIG. 6 is a front elevation of another embodiment of an output device of the present invention.

FIG. 7 is a schematic view of one other embodiment of a system of the present invention.

FIG. 8 is a schematic view of one further embodiment of the system presented in FIG. 7.

FIG. 9 is a block diagram illustration of one embodiment of a method in accordance with the present invention.

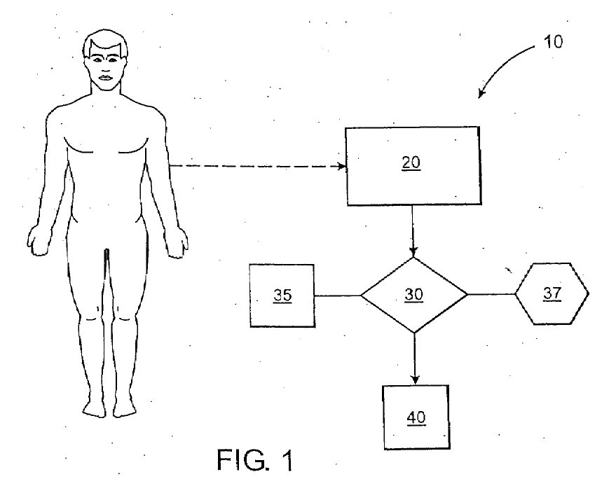

Like reference numerals refer to like parts throughout the several views of the drawings.

### DETAILED DESCRIPTION OF THE PREFERRED EMBODIMENT

While this invention is susceptible of embodiment in many different forms, there is shown in the drawings and will herein be described in detail at least one specific embodiment, with the understanding that the present disclosure is to be considered as an exemplification of the principles of the invention and is not intended to limit the invention to the embodiment illustrated.

Once again, as indicated above, the present invention is directed to a system, generally as shown at 10, for detecting an uncontrollable change in a person's physiology. While person's have been known to control and/or mask measurable changes in certain bodily functions, for example, pulse and/or respiration rates, under numerous and varied conditions such as physical stress, exertion, etc., it is also known that certain sensations can result in measurable changes in one or more of these bodily functions which are involuntary or uncontrollable. For example, it is known that a person experiencing the sensation of fear exhibits measurable changes in certain bodily functions which cannot be controlled or masked. Similarly, it is known that a person experiencing the sensation of an orgasm exhibits measurable changes in several bodily functions which are also beyond the person's ability to control or mask. The system 10 of the present invention permits the detection of these uncontrollable changes in bodily function(s), and as a result, it allows detection of a particular sensation which a person is experiencing, including, by way of example only, fear or orgasm.

The system 10 comprises a sensor assembly, generally illustrated at 20, disposed in a monitoring relation to the person. The sensor assembly 20 comprises at least one sensor structured to monitor at least one preselected bodily function of the person during a baseline monitoring period. The baseline monitoring period, in one preferred embodiment, comprises a period when the person is not exposed to any stimuli which may alter the normal value of the preselected bodily function during the baseline monitoring period. More in particular, the person should be in their normal state of health and should be at rest and not engaged in any physical or mental activity, or exposed to any excessive environmental conditions such as lighting, temperature, or noise levels, such that the data obtained during the baseline monitoring period establish the normal values of the preselected bodily function for that person. In one preferred embodiment of the present invention, the sensor assembly 20 comprises a plurality of sensors structured to monitor a plurality of preselected bodily functions of the person during the baseline monitoring period, thereby establishing normal values for a plurality of preselected bodily functions for that person.

The sensor assembly 20 is further structured to monitor at least one preselected bodily function of the person being monitored during an activity monitoring period. The activity monitoring period is any period of time during which the person is exposed to a particular form of stimulation including, by way of example only, the period of time during which a person is engaged in sexual activity, preferably including at least an amount time both before and after the person experiences the sensation of an orgasm. In one preferred embodiment of the present invention, the sensor assembly 20 comprises a plurality of sensors structured to monitor a plurality of preselected bodily functions during the activity monitoring period. Yet another embodiment provides that the sensor assembly 20 is structured to monitor one or more of the plurality of preselected bodily functions during each of a plurality of activity monitoring periods.

The preselected bodily functions of the person which may be monitored by the sensor assembly 20 of the present invention may include, but are in no way limited to, blood pressure, pulse, temperature, respiration, muscular contractions, caloric expenditure, blood oxygen level, release of sweat and/or other components such as endorphins, pheromones, adrenaline, etc., as well as sound waves generated either vocally or via pulse, heart beat, and/or respiration of the person being monitored. It is understood that other bodily functions exhibit uncontrollable changes when a person experiences certain sensations, and the scope of the system 10 of the present invention encompasses monitoring such other preselected bodily functions to allow detection of other sensations which the person may experience.

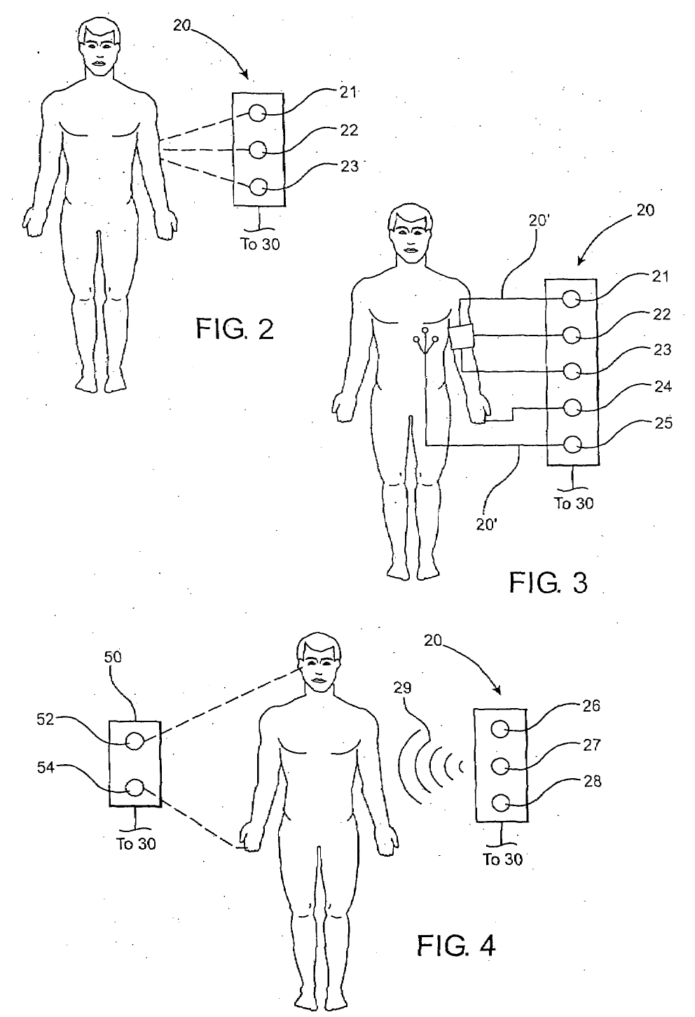

As indicated above, the sensor assembly 20 of the present invention is structured to detect at least one, but preferably a plurality of preselected bodily functions, and as such, the sensor assembly 20 comprises at least one, but preferably a plurality of detectors, each of which is disposed in a communicative relation to the person being monitored. As illustrated in FIG. 2, one preferred embodiment of the sensor assembly of the present invention comprises a temperature detector or thermal sensor 21, a blood pressure detector 22, and a pulse rate detector 23, such that systolic blood pressure, peristaltic blood pressure, and mean blood pressure may al be detected. It is understood, however, that other embodiments of the sensor assembly 20 of the present invention may comprise either additional or alternate detectors, as illustrated in FIGS. 3 and 4, respectively. As just one example, at least one embodiment of the present invention may employ a detector for measurement of blood sugar levels, such as are important to the millions of people suffering from some form of diabetes.

More in particular, FIG. 3 illustrates the sensor assembly 20 of FIG. 2 further comprising an oxygen detector or oximeter 24, structured to monitor the person's blood oxygen level, and an electrocardiograph (“EKG”) detector 25, structured to monitor the person's cardiovascular activity during the baseline and activity monitoring periods, of course, it will be appreciated that EEG and/or ECG detectors may be employed in the present invention as well. Alternately, as illustrated in FIG. 4, the sensor assembly 20 may comprise a respiration rate detector 26, an electromyograph detector 27, structured to monitor a frequency of muscular contraction of the person, and a caloric expenditure detector or calorimeter 28, structured to monitor the amount of calories expended by the person during a baseline monitoring period as well as during an activity monitoring periods, and more in particular, during at least a portion thereof.

In at least one embodiment, the sensor assembly 20 of the present invention comprises one or more sensor to monitor eye activity, such as, by way of example only, pupil dilation, sudden eye movement, focus, etc.

One further embodiment of the present invention may be utilized to measure the amount of energy required to operate and monitor a person at different times, i.e., a person may have more active monitored parameters when in an excited state resulting in a greater draw and usage of energy than when the person is in a normal, stable state. The system may also be utilized to monitor the difference in power consumption between different users. It will be appreciated that the system of the present invention may be powered by any of a variety of sources, including but not limited to alternating current electricity, direct current electricity, either rechargeable or disposable, solar power, kinetics, just to name a few.

In the embodiment illustrated in FIG. 3, the plurality of detectors 21 through 25 physically engage the person and are structured to transmit electrical, optical, audible, and/or other signals to the sensor assembly 20 via a plurality of signal carriers 20′ which may include, by way of example only, electrical wires or fiberoptic cables, thereby establishing a data communicative relationship with the sensor assembly 20.

As illustrated in FIG. 4, however, in one preferred embodiment the sensor assembly 20 is disposed in a remote monitoring relation to the plurality of detectors 21 through 25, which physically engage the person being monitored. Specifically, the “remote monitoring relation” as used herein is at least partially defined by the sensor assembly 20 being disposed in a wireless data communicative relationship with the plurality of detectors, including at least detectors 21 through 28, via one or more remote signals 29 which are transmitted between the plurality of detectors 21 through 28 and the sensor assembly 20 without requiring a physical connection between the sensor assembly 20 and the plurality of detectors 21 through 28, such as, by way of example only, an electrical wire or a fiberoptic cable.

For example, an infrared signal may be utilized to remotely and non-intrusively monitor and transfer data corresponding to the person's body temperature via the temperature detector or thermal sensor 21, without requiring a physical wired connection between the temperature detector or thermal sensor 21 and the sensor assembly 20. As another example, a sound or light wave may be remotely and non-intrusively directed towards the person's body and the person's blood pressure may be remotely monitored and transferred from the blood pressure detector 22 to the sensor assembly 20 via a remote Doppler signal, resulting from the Doppler effect on the reflected sound or light. Yet another example may include various optical signals transmitted to and/or from the person's body, either directly or to one or more detectors interconnected thereto, which allows the sensor assembly 20 to remotely monitor any number of other preselected bodily functions.

The remote monitoring relation of the sensor assembly 20 with the person may be particularly desirable in certain applications wherein physical interconnection between the plurality of detectors 21 through 28 and the sensor assembly 20, such as by electrical wires or fiberoptic cables, as illustrated in FIG. 2, may detract from the overall sensation the person is experiencing. The detraction resulting from the physical interconnection to the sensor assembly 20 may be exceptionally bothersome, for example, during sexual activity, particularly during the period of time before and after orgasm.

The system 10 of the present invention further comprises a central processing unit, generally as shown at 30, disposed in a communicating relation with the sensor assembly 20. More specifically, the sensor assembly 20 is structured to transfer a baseline data set and at least one activity data set to the central processing unit 30 which correspond to the values of at least one bodily function of the person being monitored during the baseline monitoring period and at least one activity monitoring period. In addition to the value of the bodily function being monitored, each baseline and/or activity data set may further comprise, by way of example only, an identifier of the bodily function, date and/or time stamps for each value of the bodily function obtained, and other identifying information as may be necessary to permit efficient interpretation of the baseline and/or activity data set. In one embodiment, at least one biometric parameter of the person, as discussed in more detail below, is associated with specific baseline and activity data sets, thereby providing a means to secure access to the person's data by requiring identification and verification of the biometric parameter as a condition to access the data sets. In addition, each activity data set may comprise an audio and/or video data component. The sensor assembly 20 may transfer the baseline and activity data sets to the central processing unit 30 via any one of a number of data transmission mechanisms including, by way of example only, hard wired connections or any of the myriad of wireless transmission mechanisms utilizing remote signals 29 including, by way of example only, light and/or sound waves. Further, in one preferred embodiment, the sensor assembly 20 is structured to transfer the baseline and activity data sets to the central processing unit 30 as a continuous data stream thereby approximating real time data which the central processing unit 30 interprets.

Of course, in accordance with at least one previously described embodiment, the sensor assembly 20 may be structured to transfer a plurality of baseline data sets and a plurality of activity data sets to the central processing unit 30 wherein each of the plurality of baseline and activity data sets corresponds to a different one of a plurality of bodily functions being monitored during the baseline and activity monitoring periods. In yet another embodiment, also described above, the sensor assembly 20 is structured to transfer a plurality of activity data sets to the central processing unit 30, wherein each of the plurality of activity data sets corresponds to at least one bodily function being monitored during each of a different one of a plurality of activity monitoring periods. In at least one further embodiment, the sensor assembly 20 is structured to transfer a plurality of activity data sets each corresponding to one of a plurality of bodily functions being monitored during each of different ones of a plurality of activity monitoring periods.

The central processing unit 30 of the present invention is structured to interpret at least each baseline data set and each activity data set transferred by the sensor assembly 20 and to detect a predetermined change in the preselected bodily function between the baseline monitoring period and the activity monitoring period for which each activity data set corresponds. As such, the central processing unit 30 comprises a data memory module 35 which is structured to store at least one baseline data set, and a value or range of values of a predetermined change corresponding to at least one preselected bodily function. In one preferred embodiment, the central processing unit 30 comprises a data memory module 35 structured to store a plurality of values or ranges of values of a predetermined change each corresponding to a different one of a plurality of preselected bodily functions. The central processing unit 30 is further structured to compare each activity data set with the value or range of values of the predetermined change for the preselected bodily function corresponding to the activity data set, and to detect each predetermined change therefrom. The central processing unit 30, in one preferred embodiment, is further structured to identify the magnitude or intensity of each predetermined change detected.

In addition to storing the plurality of values or ranges of values of each predetermined change, the data memory module 35 of the present invention may also be structured to store at least select ones of the baseline data sets and the plurality of activity data sets transferred to the central processing unit 30 by the sensor assembly 20. Further, the data memory module 35 of the present invention may be structured to store the baseline and activity data sets which correspond to plurality of persons. In addition, the data memory module 35 may be structured to store the results for one or more of the predetermined changes detected by the central processing unit 30, either alone or in combination with the corresponding activity data set in which the predetermined change was detected.

At least one embodiment of the central processing unit 30 of the present invention comprises an external data transmission port 37 structured to transmit at least one but preferably a plurality of baseline and activity data sets to an external data processing device, such as, by way of example only, a personal computer, laptop computer, or personal digital assistant, to provide additional data storage capacity and/or to facilitate further processing of the plurality of baseline and activity data sets, either in real time or at a future time selected by the user. As noted above with respect to the data memory module 35, the external data processing device may be structured to store, in addition to the baseline data set and the plurality of activity data sets, the results for one or more of the predetermined changes detected, either alone or in combination with the corresponding activity data set in which the predetermined change was detected. Thus, the external data processing device, for example, may be utilized to facilitate comparison of various activity data sets collected during a plurality of discreet periods of time, occurring, perhaps, over a period of years, which will allow the user to compare the intensity of the predetermined changes detected under a variety of different circumstances, as well as to identify trends and/or potential shifts in the value or range of values of the predetermined change in the preselected bodily functions being monitored.

In one embodiment of the system 10 of the present invention, the central processing unit 30 is structured to detect a predetermined change in the blood pressure of the person being monitored, and more specifically, an increase in blood pressure. For example, the predetermined change may correspond to an increase in the person's blood pressure in a range of between approximately 30 to 40 millimeters of mercury. In another embodiment, the central processing unit 30 is structured to detect a predetermined change in the pulse rate of the person being monitored. Once again, by way of example only, the predetermined change may be an increase in pulse rate in a range of between approximately 20 to 30 beats per minute. In yet one other embodiment, the central processing unit 30 is structured to detect a predetermined change in body temperature such as, again, by way of example only, an increase in a range of between approximately 0.75 to 1.25 degrees Fahrenheit.

Additional predetermined changes which the central processing unit 30 of the present invention may be structured to detect may include, but are not limited to, an increase in frequency of a muscular contraction, and in particular a muscular contraction in at least one pelvic muscle, wherein a predetermined change may be in a range of, for example, between approximately 1.0 to 1.5 cycles per second, or an increase in an amount of caloric expenditure, wherein a predetermined change may be, once again by way of example, in a range of between approximately 450 to 500 calories, during at least a portion of the specified period, such as, during an orgasm.

It is understood that the aforementioned ranges of values for predetermined changes are solely for illustrative purposes. In practice, the value or range of values for a predetermined change of a bodily function may vary from person to person depending on such factors as their respective ages, weights, health, etc., and may be higher or lower or encompass a broader or narrower ranges between different persons. In addition, the value or range of values for a predetermined change of a particular bodily function may vary for the same person depending on such factors as their age, level of physical activity, stress, health, etc., and once again may be higher or lower or encompass a broader or narrower range.

Thus, in at least one embodiment of the present invention, the value or range of values of the predetermined change of a bodily function may be set and/or reset by user input to the central processing unit 30. In addition, the central processing unit 30 may be further structured and disposed to accept and store the value or range of values of the predetermined change of one or more bodily functions for a plurality of users, which may be accessed by each user, for example, via a plurality of password protected storage areas, or thorough identification and verification of at least one biometric parameter of the person, as indicated above and described in more detail below. Alternatively, the central processing unit 30, and more specifically, the data memory module 35, may be programmed with one or more preset values or range of values of the predetermined change of one or more bodily functions. Such preset values may, for example, correspond to one or more of the age, sex, weight, etc., of the person being monitored.

While the central processing unit 30 of the present invention may be structured to detect any one of the predetermined changes described above, in at least one preferred embodiment, the central processing unit 30 is structured to detect a plurality of predetermined changes, such as, by way of example only, the predetermined changes in blood pressure, pulse rate, and temperature of the person being monitored, each of which are known to be indicative of a person experiencing the sensation of an orgasm. Thus, by structuring the central processing unit 30 to detect a plurality of predetermined changes known to be indicative of a person experiencing a particular sensation, the ability and accuracy of the system 10 of the present invention to detect when the person is experiencing such a sensation is greatly improved.

At least one preferred embodiment of the present invention further comprises an output device, as generally shown at 40, which is operatively associated with the central processing unit 30. The output device 40, in one embodiment, is structured to generate an indication upon detection of a predetermined change in at least one of the preselected bodily functions by the central processing unit 30. In one preferred embodiment, the output device 40 is structured to generate an indication upon detection of a predetermined change in each of a plurality of predetermined bodily functions being monitored during an activity monitoring period by the central processing unit 30.

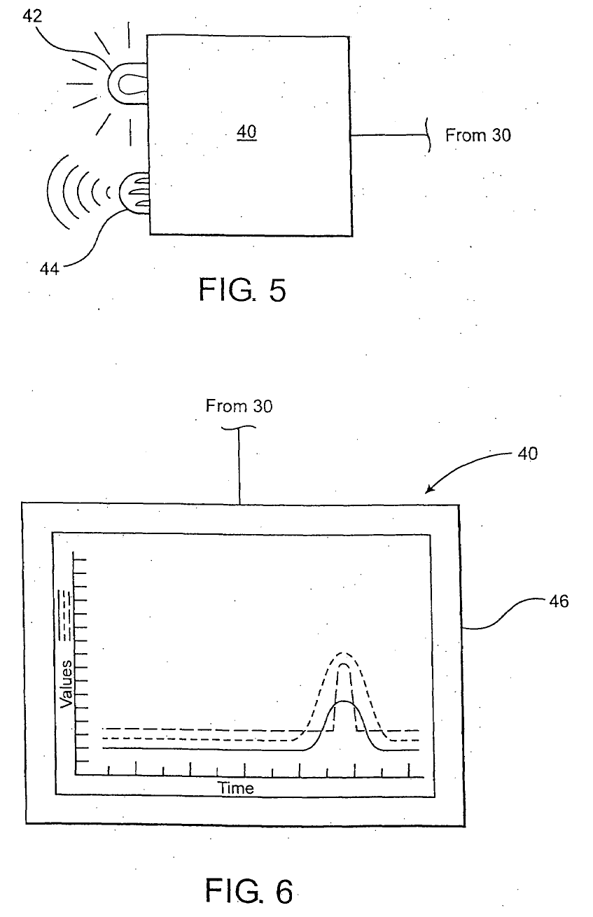

The indication may comprise an audible alarm 42 and/or a visible alarm 44, as illustrated in FIG. 5, or any other means sufficient to indicate to the person, or to any observer, that the at least one predetermined change has been detected by the central processing unit 30. Thus, the output device 40 of the present invention provides the person with results which are readily understood without the assistance or interpretation of specially trained personnel.

In one other embodiment, the output device 40 may comprise a video display screen 46 on which a report is directly viewed directly, wherein the report may include a variety of information such as the preselected bodily function or functions being monitored, the values of the preselected bodily function comprising the baseline data set, the values of the preselected bodily function comprising the corresponding activity data set, the value or range of values for the predetermined change of the preselected bodily function, and the point in time in the activity monitoring period where the predetermined change is detected. FIG. 4 provides an illustration of one embodiment of such a video display screen 46. It is clear that the report displayed on the video display screen 46 could be transmitted to a printer or saved for future use in the data memory module 35 or the external data processing device via the external data transmission port 37. It is further understood that the embodiment of the present invention comprising the video display screen 46 may also comprise the audible alarm 42 and/or the visible alarm 44 described above.

As indicated above, at least one embodiment of the present invention comprises a biometric identification device, generally as shown at 50, which is disposed in a communicative relation with the central processing unit 30. The biometric identification device 50 may be utilized to obtain at least one baseline biometric parameter from a person including, by way of example only, a fingerprint pattern, an iris recognition pattern, a voice and/or other sound recognition sample, and/or other patterns obtained from the person's body such as hair pattern, skin porosity, patterns, skin structure patterns, just to name a few. For example, as illustrated in FIG. 4, the biometric identification device may comprise an iris scanner 52 structured to obtain a baseline iris pattern from the person during the baseline monitoring period, the baseline data set thereafter being associated and accessible only upon subsequent measuring and confirmation of a match of an active iris pattern provided by the person via the iris scanner 52. Similarly, the biometric identification device 50 may comprise a fingerprint scanner 54 to obtain a baseline fingerprint pattern of the person and for subsequent measurement and confirmation of the same. As will be appreciated, any of the numerous other biometric parameters may be measured by a corresponding detection device and incorporated into the system of the present invention. Further, at least one embodiment of the present invention may comprise a combination of a biometric identification device in combination with standard “password” or “passcode” type of security measures to limit access to the system 10 of the present invention to authorized personnel. In yet one further embodiment, the present invention may employ a physical lock, combination or key, to prevent unauthorized access to the data generated by the system.

The present invention further comprises a method for detecting an uncontrollable change in a person's physiology. The method includes monitoring at least one preselected bodily function of the person during a baseline monitoring period, transferring a baseline data set to a central processing unit, such as central processing unit 30 described above, and storing the baseline data set on the central processing unit. The method further comprises monitoring the at least one preselected bodily function during an activity monitoring period, such as may be accomplished utilizing a sensor assembly 20 as described above. In one preferred embodiment, the method of the present invention comprises monitoring a plurality of preselected bodily functions during the baseline monitoring period as well as during at least one activity monitoring period. In yet another embodiment, the present method comprises monitoring a plurality of preselected bodily functions during the baseline monitoring period and each of a plurality of different activity monitoring periods.

The method of the present invention further comprises transferring an activity data set corresponding to a preselected bodily function being monitored during an activity monitoring period to the central processing unit. In at least one embodiment, the present method comprises transferring a plurality of activity data sets, each of which corresponds to a different one of a plurality of preselected bodily functions being monitored during at least one, but preferably, during each of a plurality of different activity monitoring periods.

At least one further embodiment, the method of the present invention comprises interpreting at least one activity data set utilizing the central processing unit, and detecting a predetermined change in the preselected bodily function between the baseline monitoring period and the activity monitoring period to which the activity data set corresponds. A preferred embodiment comprises interpreting a plurality of activity data sets and detecting a predetermined change in each preselected bodily function between the baseline monitoring period and the activity monitoring period to which one of the plurality of activity data sets corresponds.

In yet another embodiment, the method of the present invention comprises interpreting at least one but preferably a plurality of activity data sets manually, by comparing the value of each of the plurality of preselected bodily functions with one or more tables containing a value or range of values for the predetermined change for each of the plurality of preselected bodily functions.

Additionally, the method of the present invention comprises generating an indication upon detection of the predetermined change in the at least one preselected bodily function. In one preferred embodiment, the method comprises generating an indication upon detection of a predetermined change in each one of a plurality of preselected bodily functions being monitored during an activity monitoring period.

At least one embodiment of the method of the present invention further comprises obtaining at least one baseline biometric parameter from the person being monitored, for example, utilizing a biometric identification device 50, as described above. In addition, the method of this embodiment includes transferring the at least one baseline biometric parameter to the central processing unit 30, and storing the baseline biometric parameter thereon. This embodiment further comprises identifying the person via the biometric identification device 50 such as, for example, an iris scanner 52 or a fingerprint scanner 54, as described above, by measuring at least one active biometric parameter of the person and confirming a match with the baseline biometric parameter stored on the central processing unit 30, thereby positively identifying the person.

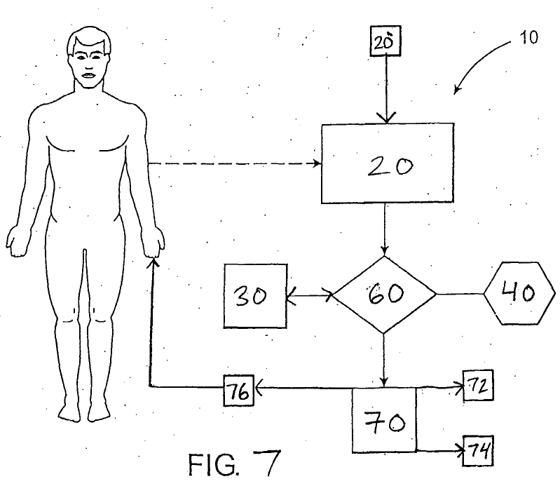

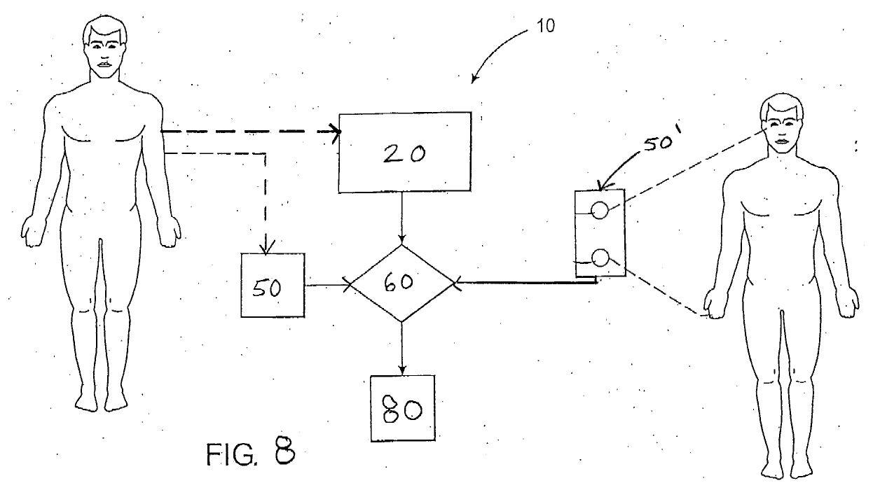

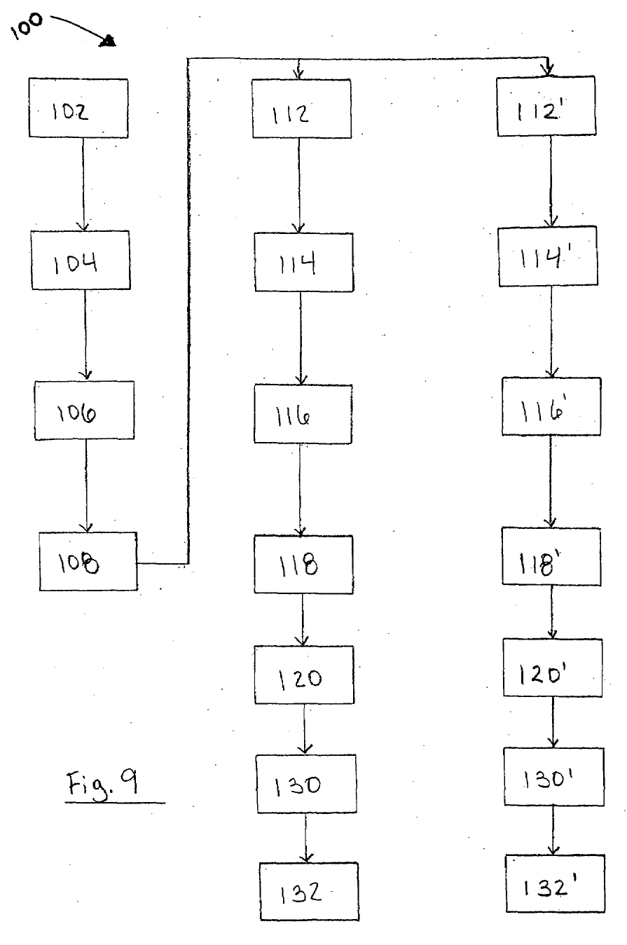

A further embodiment of the present invention comprises a system, also as generally shown at 10 in FIGS. 7 and 8, for the detection, measurement, and communication of one or more physiological property such as may be monitored, detected, and measured from a person or another living organism. As in the previously described embodiments, the system 10 of the present embodiment comprises a sensor assembly 20 which is disposed in a monitoring relation with a person or other subject, for example, another living organism such as a dog, cat, livestock, etc. Also, as above, the sensor assembly 20 of the system of the present embodiment includes at least one sensor or detector, the sensor or detector being structured to detect and measure a value of at least one preselected physiological property of a person, or other living organism, during one or more monitoring period.

In at least one embodiment, the sensor assembly 20 of the present invention employs one or more sensor or detector may be implanted directly into the person or other living organism, such as under the skin, or otherwise. As just one example, at least one embodiment of the present invention employ a microphone implanted in a person tooth such that respiration or other audible signals generated in the person's mouth or chest cavity may be monitored.

Additionally, the system 10 of the present embodiment includes a communication interface 60 which is disposed in a communicating relation with the sensor assembly 20, wherein the sensor assembly 20 is structured to communicate the value of at least one preselected physiological property which is detected and measured during the monitoring period to the communication interface 60. In at least one embodiment, the communication interface 60 includes a central processing unit 30, such as in the previously disclosed embodiments of the present invention, the central processing unit 30 being disposed in a communicating relation with the sensor assembly 20, via the communication interface 60, as shown in FIG. 7.

In at least one embodiment, the central processing unit 30 of the system 10 is structured to analyze the value of the at least one preselected physiological property, and to generate at least one activation signal, which corresponds to a value of a preselected physiological property. As above, it is understood to be within the scope and intent of the present invention for the sensor assembly 20 to monitor for and detect and measure a value of each of a plurality of physiological property during one or more monitoring period. As such, the central processing unit 30 of the present embodiment of the system 10 may be further structured to analyze each of the plurality of values of each of the plurality of physiological properties obtained during the monitoring period, and to generate one or more activation signals corresponding to each of the plurality of values, or select combinations of the same.

As will be appreciated, the central processing unit 30 may be utilized to store the value of each of a plurality of physiological property during one or more monitoring period for one person, or for a plurality of person, such as, via a plurality of secure data storage units, such that the value or values may be called up at a later time for further analysis as may be deemed necessary or desirable.

The communication interface 60, in at least one embodiment of the present invention, is further disposed in a communicating relation with at least one integrated component 70. The integrated component 70 may comprise an environmental control device 72, a personal control device 76, a security control device 74, as illustrated in FIG. 7, or any other of a plurality of devices whose operation may be affected by one or more activation signal. More in particular, in the present embodiment of the system the communication interface 60 is disposed in a communicating relation with at least one integrated component 70 and is further structured to communicate at least one activation signal to the at least one integrated component 70, thereby affecting the operation of the same.

For example, in at least one embodiment, the system 10 of the present invention employs an environmental control device 72. In this embodiment, the environmental control device 72 may include, among other things, a thermostat for an air conditioner or heating unit in a home or office. As such, the activation signal may be utilized to affect the operation of the air conditioner or heater. More specifically, a set activation signal may be generated by the central processing unit 30, and communicated to the environmental control unit 72, for example, the thermostat, by the communication interface 70, so as to simply turn an air conditioning or heating unit on or off based upon a physiological value of the person being monitored, such as, for example, an external skin temperature. In at least one embodiment, the activation signal may be based on an analysis of the values or more than one physiological property monitored, detected, and measured, for example, a difference between a person's actual body temperature and an external skin temperature may be utilized to generate an activation signal to either initiate or terminate the operation of an air conditioner or heating unit.

In at least one other embodiment, the activation signal may comprise a variable activation signal, wherein the variable activation signal may cause the operation of an integrated component 70 to operate over a range of possible operating parameters. For example, in at least one embodiment, the environmental control device 72 comprises a lighting system employing at least one dimmer switch. In this illustrative example, a physiological property being monitored may be a person's pupil diameter, and a value indicative of the degree of dilation may be utilized to generate a variable activation signal which is communicated to the environmental control device 72 by the communication interface, thereby causing the dimmer to increase or decrease the light output by the lighting system, in accordance with one or more preselected value of pupil dilation.

At least one further embodiment of the present invention comprises an environmental sensor assembly 20′, such as is illustrated in FIG. 7. More in particular, the environmental sensor assembly 20′ may be utilized to monitor, detect, and measure one or more parameters in the environment proximate a person utilizing the present system, i.e., being disposed in a monitoring relation to a sensor assembly 20. As such, the external environmental parameters may also be considered in combination with the values of the physiological parameters of the person being monitored so as to generate one or more activation signal by the central processing unit 30. By way of example only, the environmental parameters which may be monitored may include, but are by no means limited to, temperature, available light intensity, odor detection, audile decibel or other sound levels, particulate content of surrounding air, etc. As will be appreciated, any number of other environmental parameters may be monitored and detected, and as such, the values measured for such parameters may be incorporated into and utilized by the system 10 of the present invention.

In yet another embodiment, the communication interface 60 is further disposed in a communicating relation with at least one personal control device 76. As one example, a personal control device 76 may be structured to generate a discharge a low level electric current to the person being monitored, in the event that the value or values of the physiological properties measured indicate an imbalance in the person's nervous system wherein application of a low level electrical current has been proven beneficial.

In yet another embodiment, the communication interface 60 is further disposed in a communicating relation with at least one security control device 74. A security control device 74 may be utilized to permit or prevent access to a facility by a person wherein the measured value of one or more physiological property is out of a specified range, indicative of fear, deception, or other imbalance which may be exhibited by a person posing a threat to themselves or the occupants of such facility. As another example, a security personal control device 76 may be structured to permit or prevent access to a handgun or other weapon, once again, based upon values which may be indicative of whether or not a person is exhibiting rational behavior and judgment. In yet another example, an indication of fear, excitement, or other sudden change in a person's physiological properties may be utilized to trigger an alarm, activate a surveillance system, such as a video monitoring device, or any combination of the foregoing.

A further embodiment of the system for the detection, measurement, and communication of physiological properties includes at least a first biometric identification device 50 which is structured to measure at least one biometric parameter so as to positively identify a person or subject to be disposed in a monitoring relation with a sensor assembly 20, as illustrated in FIG. 8. In this embodiment, the communication interface 60 is further structured and disposed in a communicating relation with a third party monitoring device, such as is shown at 80. More in particular, the communication interface 60 in this embodiment is structured to transmit each value of one or more preselected physiological properties obtained during a monitoring period to the third party monitoring device 80, for viewing and/or analysis by one or more party, i.e., a party not being monitored by a sensor assembly 20 in accordance with the present invention. In order to protect against unwanted or unauthorized interception and monitoring of a subject's physiological properties via such a third party monitoring device 80, at least one embodiment of the present invention further comprises a second biometric identification device 50′ which is structured to measure at least one biometric parameter to positively identify an observer prior to permitting access to the third party monitoring device 80. Thus, the second biometric identification device 50′ can be utilized to permit access to the preselected physiological property values of the person or subject disposed in a monitoring relation with the sensor assembly 20.

As will be appreciated from the foregoing, at least one embodiment of the present system 10 may be utilized to allow a plurality of person's to be disposed in a monitoring relation with a sensor assembly 20, and for the value or values of one or more physiological property corresponding to each person or subject participating to be shared amongst the plurality of persons via a network of systems 10, each comprising a communication interface 60 disposed in a communicating relation with a corresponding plurality of third party monitoring devices 80.

A method, as at 100, for detecting, measuring, and communicating physiological properties in conjunction with the above-referenced system 10 comprises the following steps. To begin, the method 100 comprises a step of monitoring 102 a person or subject with the sensor assembly 20 during a baseline monitoring period. The present method 100 also includes detecting 104 one or more preselected physiological property via the sensor assembly 20 during the baseline monitoring period, and measuring 106 a baseline value of the one or more physiological property during said baseline monitoring period. The method 100 also includes communicating 108 the baseline value or values of the one or more physiological property measured during the baseline monitoring period to a communication interface, such as previously disclosed at 60.

Once the baseline values of one or more physiological property have been detected 104, measured 106, and communicated 108 to the communication interface 60, the present method further includes monitoring 112 and 112′ the person with the sensor assembly during one or more active monitoring period, additional active monitoring periods being represented by the primed reference characters. Once again, as in the baseline monitoring period, the present invention includes detecting 114 and 114′ one or more preselected physiological property via the sensor assembly 20 during the active monitoring period, and measuring 116 and 116′ an active value of one or more preselected physiological property which is detected during the active monitoring period. Also, the present method comprises communicating 118 and 118′ the active value or values of the one or more preselected physiological property measured during the active monitoring period to the communication interface 60.

After measuring 106, 116, and 116′ the baseline and active values of one or more preselected physiological property and communicating 108, 118, and 118′ the same to the communication interface 60, the method 100 of the present invention comprises analyzing 120 and 120′ the baseline value and the corresponding active value for each of the preselected physiological properties detected during the baseline and active monitoring periods. Finally, the present method includes generating 130 and 130′ at least one activation signal wherein the activation signal is at least partially defined by a predetermined variance between a baseline value and an active value of at least one of the plurality of preselected physiological properties.

At least one further embodiment of the method of the present invention comprises communicating 132 and 132′ at least one activation signal to an integrated component, thereby affecting the operation of the same, as described above.

Since many modifications, variations and changes in detail can be made to the described preferred embodiment of the invention, it is intended that all matters in the foregoing description and shown in the accompanying drawings be interpreted as illustrative and not in a limiting sense. Thus, the scope of the invention should be determined by the appended claims and their legal equivalents.

Now that the invention has been described,

What is claimed is:

1. A system for the detection, measurement, and communication of physiological properties, comprising:
a sensor assembly disposed in a monitoring relation with a subject,
a first biometric identification device structured to measure at least one biometric parameter to positively identify the subject disposed in said monitoring relation with said sensor assembly,
said sensor assembly comprising a plurality of sensors disposed in an interchangable relation to said sensor assembly,
each of said plurality of sensors structured to detect and measure a value of a different one of each of a plurality of preselected physiological properties of the person during a monitoring period,
a communication interface disposed in a communicating relation with said sensor assembly, said sensor assembly structured to communicate each said value of said different ones of said plurality of preselected physiological properties to said communication interface during said monitoring period,
said communication interface comprising a central processing unit disposed in said communicating relation with said sensor assembly,
said central processing unit being structured to analyze each said value of said different ones of said plurality of preselected physiological properties and to generate at least one activation signal corresponding to said values,
said communication interface further disposed in a communicating relation with at least one integrated component, said communication interface being further structured to communicate said activation signal to said at least one integrated component,
said communication interface further structured and disposed in a communicating relation with a third party monitoring device, and
said communication interface structured to transmit each said value of said different ones of said plurality of preselected physiological properties during said monitoring period to said third party monitoring device, and
a second biometric identification device structured to measure at least one biometric parameter to positively identify an observer prior to permitting access to said third party monitoring device.

2. The system as recited in claim 1 wherein at least one of said plurality of sensors structured to detect and measure a value of an environmental parameter proximate the person during said monitoring period.

3. The system as recited in claim 1 wherein said integrated component comprises an environmental control device.

4. The system as recited in claim 3 wherein said environmental control device comprises a temperature control unit.

5. The system as recited in claim 3 wherein said environmental control device comprises a lighting control unit.

6. The system as recited in claim 1 wherein said integrated component comprises a personal control device.

7. The system as recited in claim 6 wherein said personal control device comprises an electrical generator structured to apply an electrical current to the person.

8. The system as recited in claim 6 wherein said personal control device comprises a binaural generator structured to transmit an audible binaural beat to the person.

9. The system as recited in claim 1 wherein said activation signal comprises a set command.

10. The system as recited in claim 1 wherein said activation signal comprises a variable command.

11. The system as recited in claim 1 wherein said integrated component comprises a security control device.

12. The system as recited in claim 11 wherein said security control device comprises a weapon access unit.

13. The system as recited in claim 12 wherein said security control device comprises a facility access unit.
